(19) United States
(12) Patent Application Publication
(10) Pub. No.: US 2017/0000411 A1
(43) Pub. Date: Jan. 5, 2017

(54) BIOMETRICS INFORMATION REGISTRATION METHOD, BIOMETRICS AUTHENTICATION METHOD, BIOMETRICS INFORMATION REGISTRATION DEVICE AND BIOMETRICS AUTHENTICATION DEVICE

(71) Applicant: FUJITSU FRONTECH LIMITED, Tokyo (JP)

(72) Inventor: Kazuhiro Komura, Tokyo (JP)

(21) Appl. No.: 15/266,067

(22) Filed: Sep. 15, 2016

Related U.S. Application Data
(63) Continuation of application No. PCT/JP2015/059213, filed on Mar. 25, 2015.

(30) Foreign Application Priority Data
Mar. 25, 2014 (JP) 2014-062775

(51) Int. Cl. A61B 5/00; G06K 9/00; G06F 21/32; A61B 5/1171

(52) U.S. Cl. CPC A61B 5/489; A61B 5/0077; A61B 5/1171; G06K 9/00067; G06K 9/00926; G06K 9/00087; G06F 21/32; A61B 2560/0475; A61B 2576/00; G06K 2009/00932

(57) ABSTRACT

A biometrics information registration method for causing a computer to execute a process including extracting vein data representing a vein image and a feature amount from an image obtained by an image obtainment unit; and making a storage unit store the vein data and the feature amount, wherein the feature amount includes a first feature amount representing a relationship between two of a plurality of segments obtained by vectorizing the vein image.

### CROSS-REFERENCE TO RELATED APPLICATION

This application is continuation application of International Application PCT/JP2015/059213 filed on Mar. 25 2015 and designated the U.S., the entire contents of which are incorporated herein by reference. This application is based upon and claims the benefit of priority from the prior Japanese Patent Application No. 2014-062775, filed on Mar. 25, 2014, the entire contents of which are incorporated herein by reference.

### FIELD

The embodiments of the present disclosure are related to a technique of biometrics authentication that uses vein data in order to determine whether or not the subject is a person to be authenticated.

### BACKGROUND

An existing biometrics authentication method for example conducts matching (a matching process) between matching vein data obtained from an image based on photography of the user and registered vein data which has been registered in advance, so as to determine whether or not the user is a person to be authenticated, on the basis of the degree of similarity, obtained by that matching, between the matching vein data and the registered vein data (1:1 authentication).

Besides the 1:1 authentication, another existing biometrics authentication method conducts matching between matching vein data and a plurality of pieces of registered vein data respectively so as to determine whether or not the subject is a person to be authenticated, on the basis of the highest degree of similarity among a plurality of degrees of similarity obtained by the matching (1:N authentication).

In matching that uses a lot of data such as one using vein data, a process takes a long period of time for each case, leading to a situation where an increase in the number of registered cases of pieces of registered vein data (N) lengthens the process time in 1:N authentication.

In view of this, there is a method for reducing a process time in 1:N authentication. As an example, there is a method in which a plurality of pieces of registered vein data are sorted in descending order of degree of similarity between matching feature amounts obtained from matching vein data and registered feature amounts obtained from registered vein data so that matching is conducted between the matching vein data and pieces of registered vein data that are ranked highly in the sorting (for example, Japanese Laid-open Patent Publication No. 2007-249339 and Japanese Patent No. 5363587).

### SUMMARY

A biometrics information registration method according to an embodiment of the present disclosure is a biometrics information registration method for causing a computer to execute a process including extracting vein data representing a vein image and a feature amount from an image obtained by an image obtainment unit; and making a storage unit store the vein data and the feature amount, wherein the feature amount includes a first feature amount representing a relationship between two of a plurality of segments obtained by vectorizing the vein image.

A biometrics authentication method according to an embodiment of the present disclosure is a biometrics authentication method for causing a computer to execute a process including extracting vein data representing a vein image and a feature amount from an image obtained by an image obtainment unit; narrowing down a plurality of pieces of registered vein data stored in the storage unit on the basis of a comparison result between the extracted feature amount and a registered feature amount stored in the storage unit, and obtaining a degree of similarity between the pieces of registered vein data that were narrowed down and the extracted vein data; and determining whether or not a subject is a person to be authenticated, on the basis of the obtained degree of similarity, wherein the feature amount includes a first feature amount representing a relationship between two of a plurality of segments obtained by vectorizing the vein image.

A biometrics information registration device according to an embodiment of the present disclosure is a biometrics information registration device including a feature amount extraction unit for extracting vein data representing a vein image and a feature amount from an image obtained by an image obtainment unit; and a feature amount registration unit for making a storage unit store the vein data and the feature amount extracted by the feature amount extraction unit, wherein the feature amount includes a first feature amount representing a relationship between two of a plurality of segments obtained by vectorizing the vein image.

A biometrics authentication device according to an embodiment of the present disclosure is a biometrics authentication device including a feature amount extraction unit for extracting vein data representing a vein image and a feature amount from an image obtained by an image obtainment unit; a matching unit for narrowing down a plurality of pieces of registered vein data stored in the storage unit on the basis of a comparison result between the feature amount extracted by the feature amount extraction unit and a registered feature amount stored in the storage unit, and obtaining a degree of similarity between the pieces of registered vein data that were narrowed down and the vein data extracted by the feature amount extraction unit; and a determination unit for determining whether or not a subject is a person to be authenticated, on the basis of the degree of similarity obtained by the matching unit, wherein the feature amount includes a first feature amount representing a relationship between two of a plurality of segments obtained by vectorizing the vein image.

A non-transitory computer-readable record medium according to an embodiment of the present disclosure which records a program for causing a computer to execute a process including extracting vein data representing a vein image and a feature amount from an image obtained by an image obtainment unit; and making a storage unit store the extracted vein data and feature amount, wherein the feature amount includes a first feature amount representing a relationship between two of a plurality of segments obtained by vectorizing the vein image.

A non-transitory computer-readable record medium according to an embodiment of the present disclosure which records a program for causing a computer to execute a process including extracting vein data representing a vein image and a feature amount from an image obtained by an image obtainment unit; narrowing down a plurality of pieces of registered vein data stored in the storage unit on the basis of a comparison result between the extracted feature amount and a registered feature amount stored in the storage unit, and obtaining a degree of similarity between the pieces of registered vein data that were narrowed down and the vein data extracted by the feature amount extraction unit; and determining whether or not a subject is a person to be authenticated, on the basis of the obtained degree of similarity, wherein the feature amount includes a first feature amount representing a relationship between two of a plurality of segments obtained by vectorizing the vein image.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 shows a biometrics information registration device according to an embodiment of the present disclosure;

FIG. 2 is a flowchart for a biometrics information registration method;

FIG. 3 shows an example of a picked-up image;

FIG. 4 shows an example of vein data;

FIG. 5 shows an example of data stored in a storage unit;

FIG. 6 shows a biometrics authentication device according to an embodiment of the present disclosure;

FIG. 7 is a flowchart for a biometrics authentication method;

FIG. 8A shows a process of narrowing down pieces of vein data;

FIG. 8B shows a process of narrowing down pieces of vein data;

FIG. 8C shows a process of narrowing down pieces of vein data;

FIG. 9A shows a determination process;

FIG. 9B shows a determination process;

FIG. 9C shows a determination process;

FIG. 10 is a flowchart for a feature amount extraction process;

FIG. 11A shows an example of a division pattern;

FIG. 11B shows an example of a division pattern;

FIG. 12 shows an example of a segment;

FIG. 13 is a flowchart for a feature amount calculation process;

FIG. 14A shows a first feature amount;

FIG. 14B shows a first feature amount;

FIG. 14C shows a first feature amount;

FIG. 15A shows an example of a segment;

FIG. 15B shows an example of a segment;

FIG. 15C shows an example of a segment;

FIG. 15D shows an example of a segment;

FIG. 16A shows a second feature amount;

FIG. 16B shows a second feature amount;

FIG. 16C shows a second feature amount;

FIG. 17 shows a third feature amount;

FIG. 18 shows a fourth feature amount;

FIG. 19A shows a fifth feature amount;

FIG. 19B shows a fifth feature amount;

FIG. 19C shows a fifth feature amount;

FIG. 19D shows a fifth feature amount;

FIG. 20A shows a sixth feature amount;

FIG. 20B shows a sixth feature amount;

FIG. 20C shows a sixth feature amount;

FIG. 20D shows a sixth feature amount;

FIG. 21A shows a seventh feature amount;

FIG. 21B shows a seventh feature amount; and

FIG. 22 shows an example of hardware of a biometrics information registration device or a biometrics authentication device.

### DESCRIPTION OF EMBODIMENTS

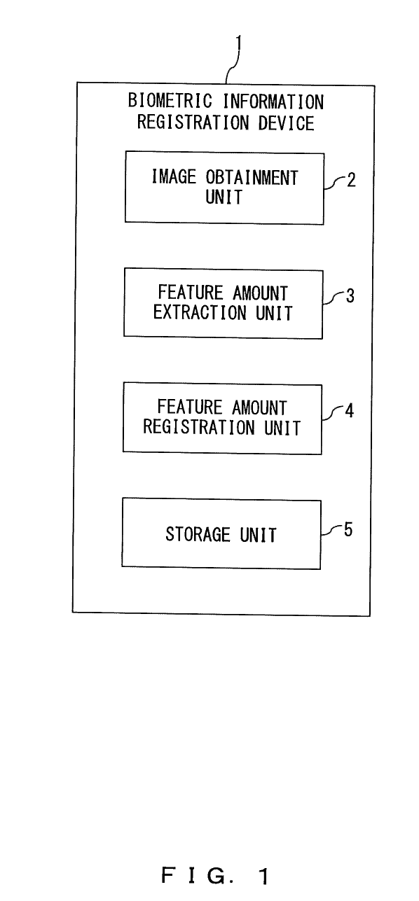

FIG. 1 shows a biometrics information registration device according to an embodiment of the present disclosure.

A biometrics information registration device 1 shown in FIG. 1 includes an image obtainment unit 2, a feature amount extraction unit 3, a feature amount registration unit 4 and a storage unit 5. The image obtainment unit 2 and the storage unit 5 may be provided outside the biometrics information registration device 1.

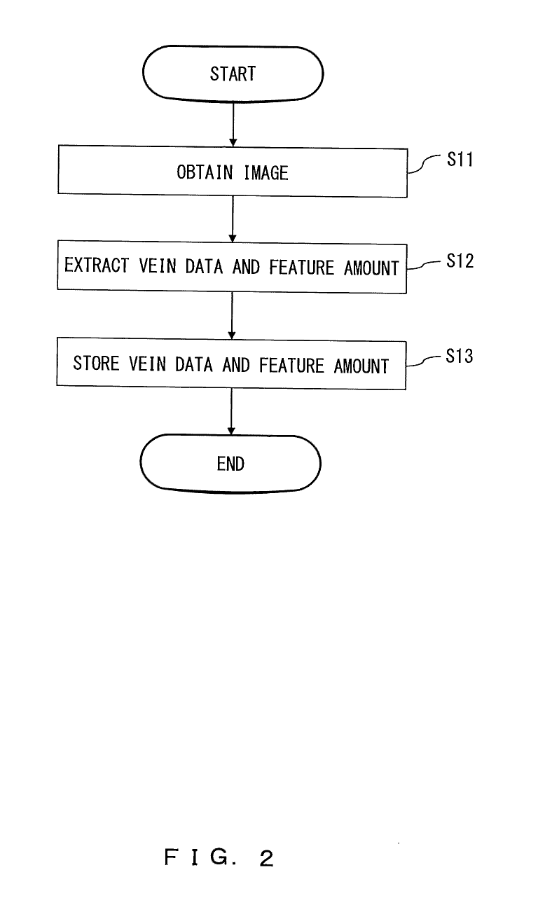

FIG. 2 is a flowchart explaining a biometrics information registration method.

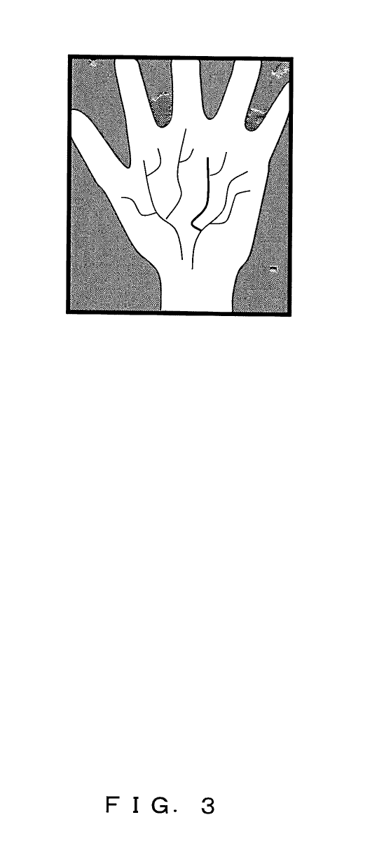

First, the image obtainment unit 2 obtains a picked-up image of the hand of the user (S11). For example, the image obtainment unit 2 is an image pickup device and photographs the hand of the user by using a single-panel image pick-up element and the RGB color filters of a Bayer array. Also, the image obtainment unit 2 casts near-infrared rays on the hand of the user so as to pick up the reflected light. Because hemoglobin in erythrocytes, which flow through veins, absorb near-infrared rays, portions containing veins, which issue reflected light with a lower intensity, are black in a picked-up image as shown in FIG. 3. The image obtainment unit 2 may further obtain an image including only the palm region of the user from a picked-up image.

Next, the feature amount extraction unit 3 extracts vein data, which represents a vein image, and the feature amount from an image obtained by the image obtainment unit 2 (S12). For example, the feature amount extraction unit 3 extracts vein data as shown in FIG. 4.

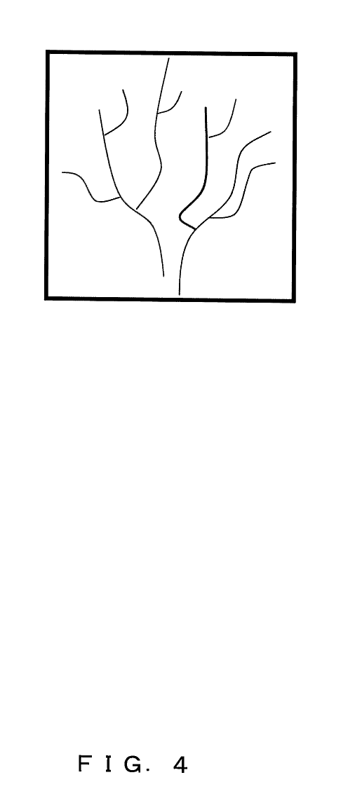

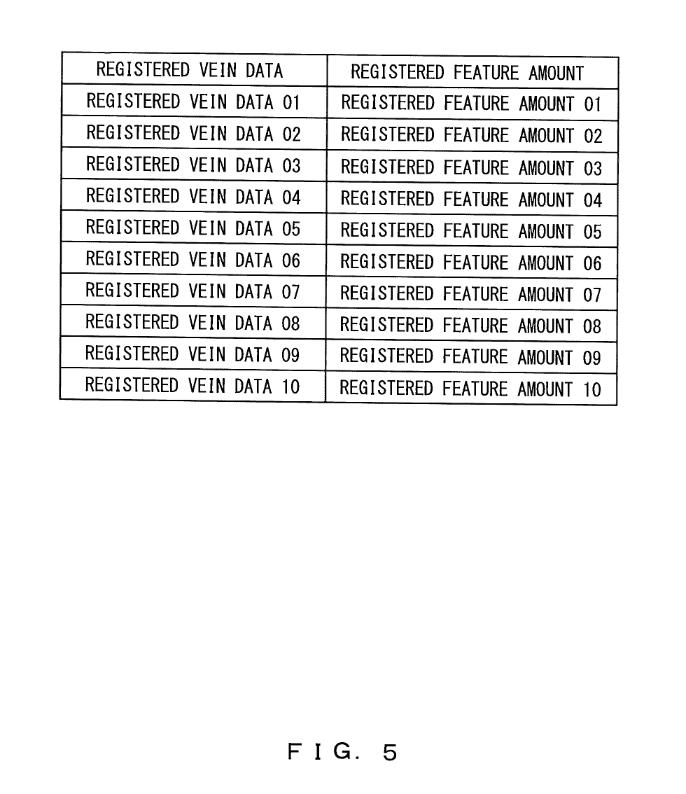

Then, the feature amount registration unit 4 makes the storage unit 5 store the vein data and feature amount extracted by the feature amount extraction unit 3 (S13). For example, as shown in FIG. 5, the feature amount registration unit 4 makes the storage unit 5 store, as pieces of registered vein data 01 through 10 and registered feature amounts 01 through 10, the vein data and the feature amounts corresponding to the vein data extracted by the feature amount extraction unit 3 for ten users.

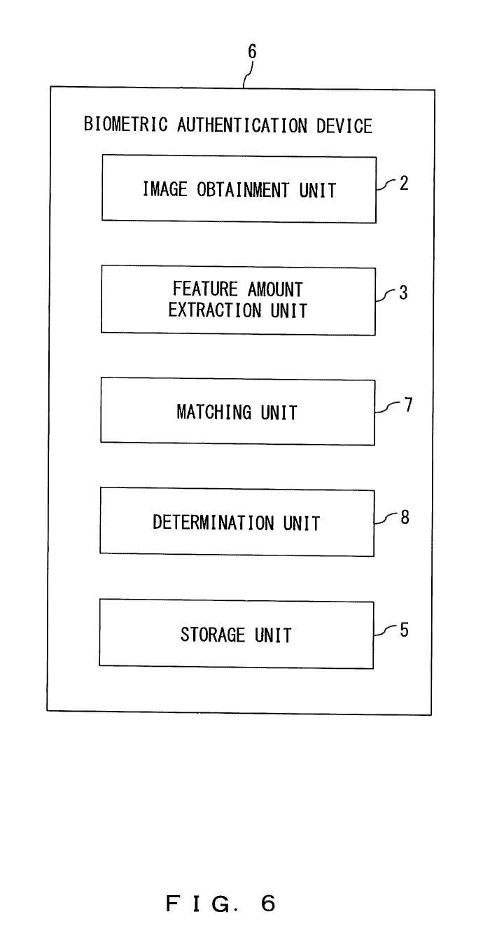

FIG. 6 shows a biometrics authentication device according to an embodiment of the present disclosure. Note that constituents similar to those in the configuration shown in FIG. 1 are denoted by the same symbols and explanations thereof will be omitted.

A biometrics authentication device 6 shown in FIG. 6 includes the image obtainment unit 2, the feature amount extraction unit 3, the storage unit 5, a matching unit 7 and a determination unit 8. Note that the image obtainment unit 2 and the storage unit 5 may be provided outside the biometrics authentication device 6.

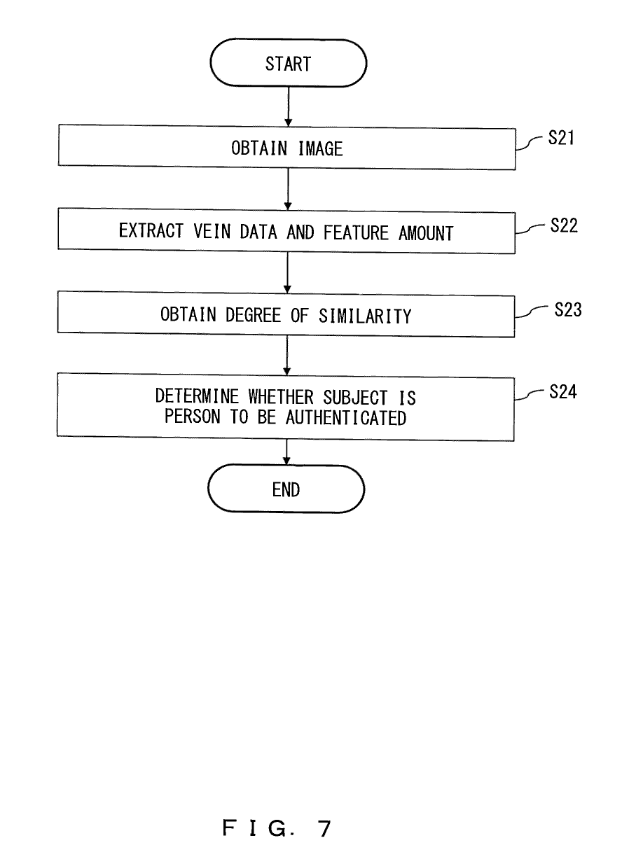

FIG. 7 is a flowchart for a biometrics authentication method.

First, the image obtainment unit 2 obtains a picked-up image of the hand of the user (S21).

Next, the feature amount extraction unit 3 extracts the vein data and the feature amount from the image obtained by the image obtainment unit 2 (S22).

Next, on the basis of a comparison result between a feature amount extracted by the feature amount extraction unit 3 and a registered feature amount stored in the storage unit 5 in advance, the matching unit 7 narrows down a plurality of pieces of registered vein data stored in the storage unit 5 in advance and obtains the degrees of similarity between the narrowed-down pieces of registered vein data and the vein data extracted by the feature amount extraction unit 3 (S23).

Then, the determination unit 8 determines whether or not the user is a person to be authenticated, on the basis of the degree of similarity obtained by the matching unit 7 (S24).

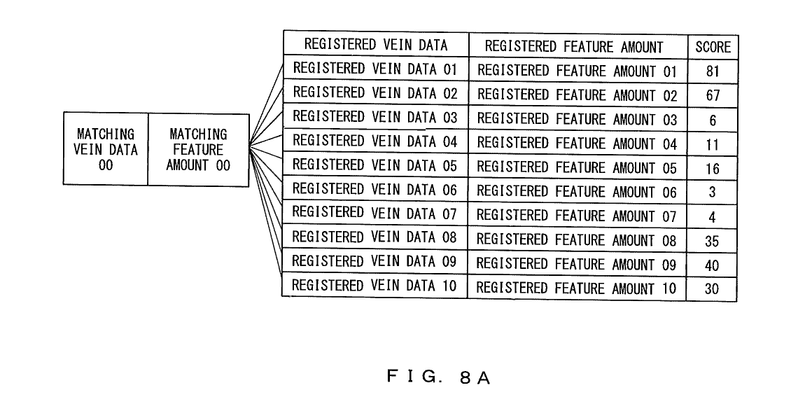

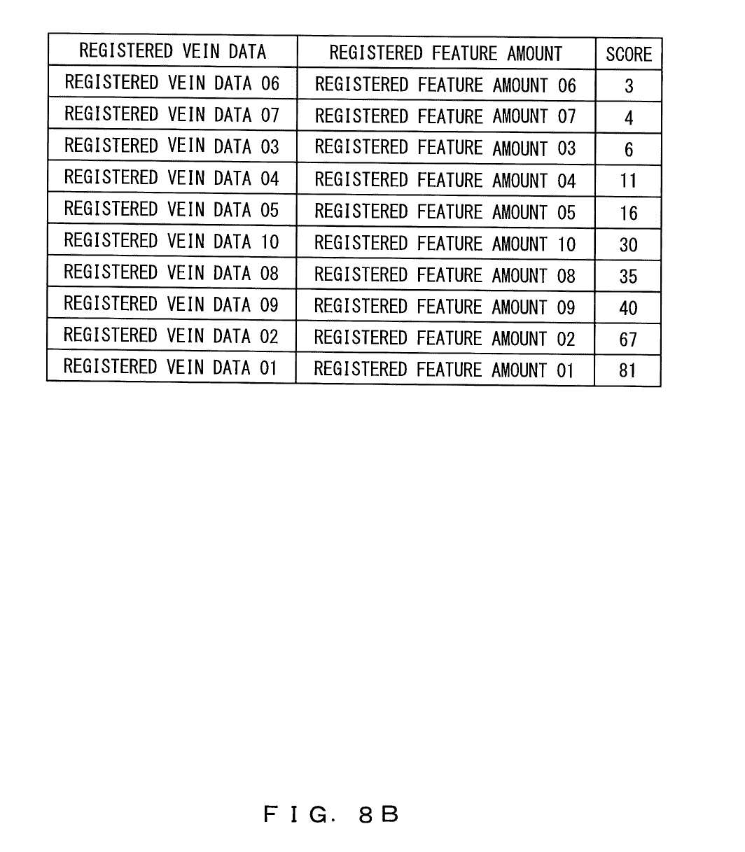

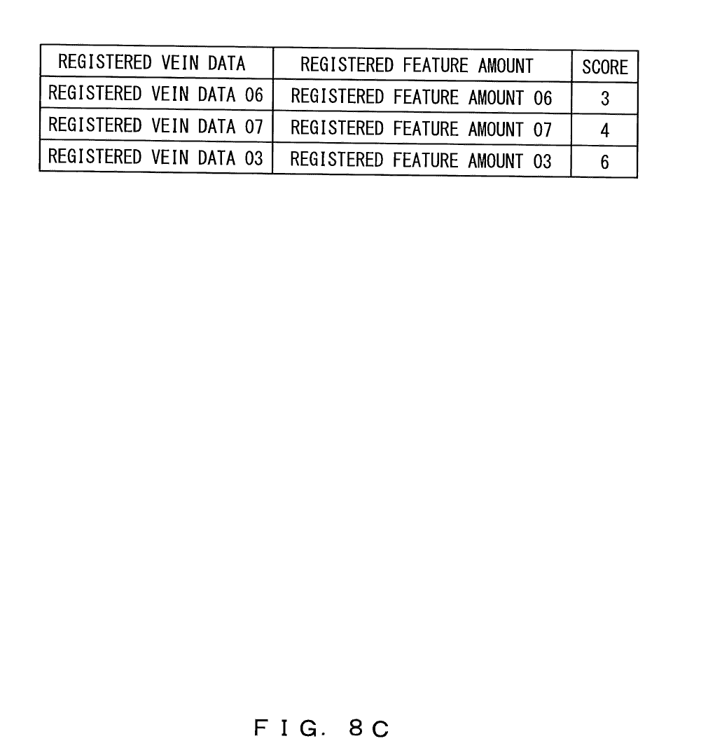

For example, the matching unit 7 makes the storage unit 5 store, as a score and together with corresponding registered vein data, the absolute value of a difference between a matching feature amount extracted by the feature amount extraction unit 3 and a registered feature amount stored in the storage unit 5. In the example shown in FIG. 8A, an absolute value 81 of a difference between matching feature amount 00 extracted by the feature amount extraction unit 3 and registered feature amount 01 stored in the storage unit 5 is stored as a score in the storage unit 5 together with registered vein data 01, an absolute value 67 of a difference between matching feature amount 00 and registered feature amount 02 is stored as a score in the storage unit 5 together with registered vein data 02, . . . , and an absolute value 30 of a difference between matching feature amount 00 and registered feature amount 10 is stored as a score in the storage unit 5 together with registered vein data 10. It is assumed that a smaller score leads to a higher possibility that the degree of similarity, corresponding to that score, between the matching vein data and the registered vein data will become higher.

Next, the matching unit 7 sorts in ascending order the scores stored in the storage unit 5. In the example shown in FIG. 8B, as a result of the sorting, the scores have been rearranged to the order of 3, 4, . . . , 81 and the pieces of registered vein data have been rearranged to the order of 06, 07, . . . , 01, accompanying the sorting.

Next, the matching unit 7 narrows down the number of pieces of registered vein data to a prescribed percentage from the top of the sorted pieces of registered vein data. In the example shown in FIG. 8C, the pieces of registered vein data have been narrowed down to pieces of registered vein data 06, 07 and 03, which account for the top 30 percent.

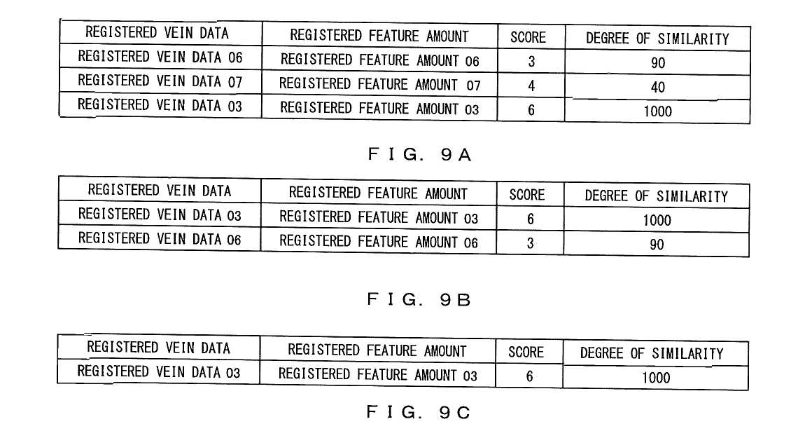

Next, the matching unit 7 obtains the degrees of similarity between matching vein data extracted by the feature amount extraction unit 3 and the narrowed-down pieces of registered vein data. In the example shown in FIG. 9A, “90” is obtained as the degree of similarity between matching vein data 00 and registered vein data 06, “40” is obtained as the degree of similarity between matching vein data 00 and registered vein data 07, and “1000” is obtained as the degree of similarity between matching vein data 00 and registered vein data 03.

Next, the matching unit 7 extracts degrees of similarity that are equal to or higher than a threshold from among degrees of similarity corresponding to the narrowed-down pieces of registered vein data, and sorts the extracted degrees of similarity in descending order. In the example shown in FIG. 9B, the degrees of similarity of “90” and “1000”, which are greater than the threshold of “50”, have been extracted, and the extracted degrees of similarity of “90” and “1000” are sorted in descending order so that they are arranged in the order of the degrees of similarity of “1000” and “90” from the top.

Next, from among the sorted degrees of similarity, the matching unit 7 obtains the highest degree of similarity as the degree of similarity for matching. In the example shown in FIG. 9C, the degree of similarity of “1000”, which is the greatest, is obtained as the degree of similarity for matching.

Next, the determination unit 8 determines that the user is a person to be authenticated when the degree of similarity obtained by the matching unit 7 is equal to or higher than the threshold.

Next, the feature amount extraction process (S12) shown in FIG. 2 and the feature amount extraction process (S22) shown in FIG. 7 will be explained.

FIG. 10 is a flowchart for a feature amount extraction process.

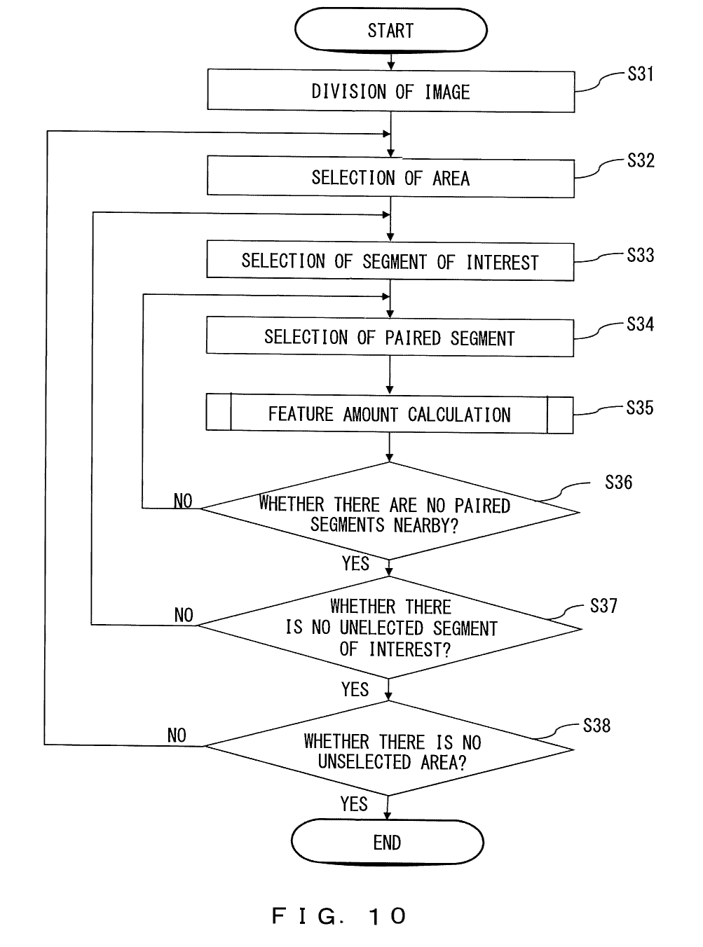

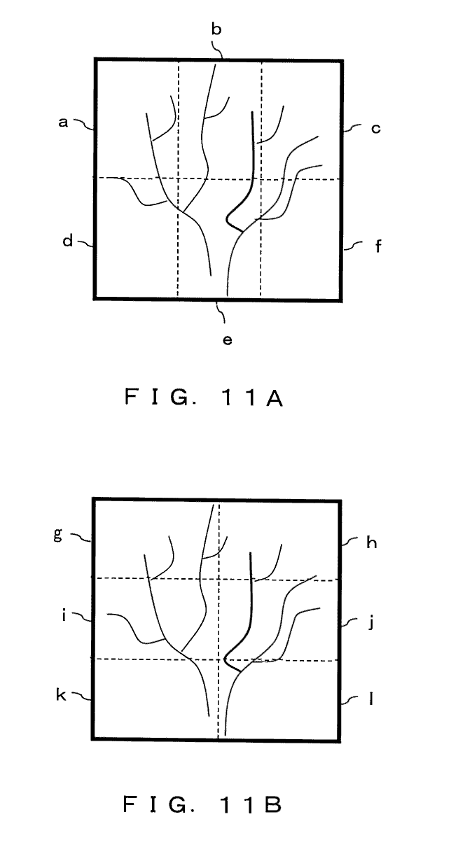

First, the feature amount extraction unit 3 divides an image obtained by the image obtainment unit 2 into a plurality of areas by a prescribed division pattern (S31). When for example the image is divided into three areas horizontally and two areas vertically (division pattern P1), the feature amount extraction unit 3 obtains six areas a through f as shown in FIG. 11A. Also, when the image is divided into two areas horizontally and three areas vertically (division pattern P2), the feature amount extraction unit 3 obtains six areas g through l as shown in FIG. 11B. There is a possibility that the division of an image as described above will prevent correct recognition of vein data located on the boundary between areas obtained by the division based on division pattern P1, whereas correct recognition is possible for division pattern P2 because vein data is not located on the boundaries between areas obtained by the division based on division pattern P2. This makes it possible for both of them to compensate for each other's lack of vein data. Also, in a case where a feature amount is extracted in an individual area obtained by dividing an image, it is more difficult to average the feature amounts than in a case where the feature amount of the entire image is extracted without dividing the image, and thus higher accuracy can be attained in extracting the feature amounts. Note that, the division patterns are not limited to that shown in FIG. 11A or FIG. 11B.

Next, the feature amount extraction unit 3 selects one of the plurality of divisional areas (S32). For example, feature amount extraction unit 3 selects area c from among six areas a through f shown in FIG. 11A.

Next, from among a plurality of segments obtained by vectorizing the vein image in the selected area, the feature amount extraction unit 3 selects a segment of interest (S33), and selects a paired segment (S34). For example, the feature amount extraction unit 3 selects segment c1 as a segment of interest and selects segment c2 as a paired segment from among segments c1 through c3 in area c as shown in FIG. 12.

Next, the feature amount extraction unit 3 calculates the feature amount (S35).

Next, the feature amount extraction unit 3 determines whether or not there are no paired segments near the segment of interest (S36), and when it is determined that there is a paired segment (No in S36), the feature amount extraction unit 3 returns to the process in S34, and when it is determined that there is not a paired segment (Yes in S36), it is determined whether or not there is a segment of interest that has not been selected in the selected area (S37). For example, when distance L between segment of interest c1 and segment c3 is equal to or longer than a threshold as shown in FIG. 12, it is determined that there are no paired segments near segment of interest c1.

Next, when it is determined that there is an unselected segment of interest (No in S37), the feature amount extraction unit 3 returns to the process in S33, and when it is determined that there are no unselected segments of interest (Yes in S37), the feature amount extraction unit 3 determines whether or not there is an unselected area (S38). For example, when all of segments c1 through c3 shown in FIG. 12 are selected as segments of interest, the feature amount extraction unit 3 determines that there are no unselected segments of interest in area c.

When it is determined that there is an unselected area (No in S38), the feature amount extraction unit 3 returns to the process in S32, while when it is determined that there are no unselected areas (Yes in S38), the feature amount extraction unit 3 terminates the feature amount extraction process.

Next, explanations will be given for the feature amount calculation process in S35 shown in FIG. 10.

FIG. 13 is a flowchart for the feature amount calculation process in S35.

First, the feature amount extraction unit 3 selects a divisional segment of interest for a segment of interest (S41).

Next, the feature amount extraction unit 3 selects a paired divisional segment for a paired segment (S42).

Next, the feature amount extraction unit 3 calculates a feature amount that represents the relationship between the divisional segment of interest and the paired divisional segment (S43).

Next, when it is determined that the segment is not the last one of the selectable paired divisional segments (No in S44), the feature amount extraction unit 3 selects the next paired divisional segment (S42), and calculates a feature amount representing the divisional segment of interest and the next paired divisional segment (S43).

Also, when it is determined that the segment is the last one of the selectable paired divisional segments (Yes in S44), the feature amount extraction unit 3 determines whether or not the segment is the last one of the selectable divisional segment of interest (S45).

Next, when it is determined that the segment is not the last one of the selectable divisional segment of interest (No in S45), the feature amount extraction unit 3 selects the next divisional segment of interest (S41), and repeats S42 through S44 to the last paired divisional segment.

When it is determined that the segment is the last one of the selectable divisional segment of interest (Yes in S45), the feature amount extraction unit 3 terminates the feature amount calculation process.

A case is assumed for example in which the image has been divided by division pattern P1 and area c has been selected as shown in FIG. 11A and segment c1 has been selected as a segment of interest c1 and segment c2 has been selected as paired segment c2 as shown in FIG. 12.

First, the feature amount extraction unit 3 obtains all end points and inflection points of segment of interest c1 and treats these points as points c11 through c16 as shown in FIG. 14A, treats point c11 as point A, treats as point B a point distant from point A on segment of interest c1 by linear distance len, and treats straight line AB passing through points A and B as divisional segment of interest AB as shown in FIG. 14B.

Next, the feature amount extraction unit 3 obtains all end points and inflection points on paired segment c2 as shown in FIG. 14A, treats these points as points c21 through c26, treats point c21 as point C while treating a point on paired segment c2 distant from point C by linear distance len as point D, and treats straight line CD passing through points C and D as paired divisional segment CD as shown in FIG. 14B.

Next, the feature amount extraction unit 3 calculates θ1=a cos(AB·CD/|AB∥CD|)*(180/π), and thereby obtains angle θ1 between divisional segment of interest AB and paired divisional segment CD as shown in FIG. 14B. Note that when area c is substituted by a two-dimensional coordinate, the coordinates of point A, point B, point C and point D are treated as (xa,ya), (xb,yb), (xc,yc) and (xd,yd) respectively. Also, it is assumed that AB·CD=(xb−xa)(xd−xc)+(yb−ya)(yd−yc) and |AB|=((xb−xa)²+(yb−ya)²)^{1/2} are satisfied and |CD|=((xd−xc)²+(yd−yc)²)^{1/2} is also satisfied.

Next, the feature amount extraction unit 3 obtains hist1_P1[Area][n] as a histogram (frequency distribution) for angle θ1. Note that P1 represents division pattern P1, [Area] represents an area after the division of the image, and [n] represents the number of grades of a histogram. For example, when the angle of 180 degrees is partitioned in units of 6 degrees so as to set 30 angle regions (grades), the counter value of the angle region including angle θ1 is incremented from among counter values sdir(0) through sdir(29) respectively corresponding to the 30 angle regions, and obtains hist1_P1[c][30]={sdir(0), sdir(1), . . . , sdir(29)} as a histogram. When for example angle θ1 calculated then is 30 degrees, the feature amount extraction unit 3 increments counter value sdir(5), which corresponds to the angle region from 30 degrees to 35 degrees, so as to obtain hist1_P1[c][30]={0, 0, 0, 0, 0, 1, 0, . . . , 0}.

Next, as shown in FIG. 14C, the feature amount extraction unit 3 treats next point c22 as point C, treats as point D a point distant from point C on paired segment c2 by linear distance len, and treats straight line CD passing through points C and D as next paired divisional segment CD.

Next, the feature amount extraction unit 3 calculates angle θ1 between divisional segment of interest AB and next paired divisional segment CD.

Next, the feature amount extraction unit 3 increments the counter value of the angle region including that angle θ1, and obtains hist1_P1[c][30]={sdir(0), sdir(1), . . . , sdir(29)} as a histogram. When for example angle θ1 calculates that time is 43 degrees, the feature amount extraction unit 3 increments counter value sdir(7), which corresponds to the angle region from 42 degrees to 47 degrees, and obtains hist1_P1[c][30]={0, 0, 0, 0, 0, 1, 0, 1 . . . , 0}.

As described above, angle θ1 between divisional segment of interest AB and paired divisional segment CD is repeatedly calculated by referring to point c11 until it becomes impossible to select paired divisional segment CD in paired segment c2. Thereafter, hist1_P1[c][30]={sdir(0), sdir(1), . . . , sdir(29)} is obtained as a histogram for each of such angles θ1.

Next, the feature amount extraction unit 3 treats next point c12 as point A, treats as point B a point distant from point A on segment of interest c1 by linear distance len, treats straight line AB passing through points A and B as next divisional segment of interest AB, treats point c21 as point C, treats as point D a point distant from point C on paired segment c2 by linear distance len, and treats straight line CD passing through points C and D as paired divisional segment CD.

Next, the feature amount extraction unit 3 calculates angle θ1, increments the counter value of the angle region including that angle θ1, and obtains hist1_P1[c][30]={sdir(0), sdir(1), . . . , sdir(29)} as a histogram.

As described above, angle θ1 is obtained for each of all the combinations between selectable divisional segments of interest AB and selectable paired divisional segments CD, so as to obtain hist1_P1[c][30]={sdir(0), sdir(1), . . . , sdir(29)} as a histogram for that angle θ1.

Next, the feature amount extraction unit 3 similarly obtains hist1_P1[a][30], hist1_P1[b][30], hist1_P1[d][30], hist1_P1[e][30] and hist1_P1[f][30] as histograms for other areas a, b, d, e and f of the image shown in FIG. 11A.

Then, the feature amount extraction unit 3 performs a normalization process on each of hist1_P1[a][30] through hist1_P1[f][30]. For example, the feature amount extraction unit 3 treats, as ALLcnt1, the sum of the total of sdir(0), sdir(1), . . . , sdir(29) of histogram hist1_P1[a][30], the total of sdir(0), sdir(1), . . . , sdir(29) of hist1_P1[b][30], . . . , and the total of sdir(0), sdir(1), . . . , dir(29) of hist1_P1[f][30], expresses the ratios of the counter values by dividing each counter value by sum ALLcnt1 in an integer as described below so as to perform a normalization process, and makes the storage unit 5 store normalized histograms hist1_P1[a][30] through hist1_P1[f][30] as the above matching feature amounts or the above registered feature amounts (first feature amount).

hist1_P1[a][30]={sdir(0)/ALLcnt1, sdir(1)/ALLcnt1, . . . , sdir(29)/A LLcnt1}
hist1_P1[b][30]={sdir(0)/ALLcnt1, sdir(1)/ALLcnt1, . . . , sdir(29)/A LLcnt1}
hist1_P1[c][30]={sdir(0)/ALLcnt1, sdir(1)/ALLcnt1, . . . , sdir(29)/A LLcnt1}
hist1_P1[d][30]={sdir(0)/ALLcnt1, sdir(1)/ALLcnt1, . . . , sdir(29)/A LLcnt1}
hist1_P1[e][30]={sdir(0)/ALLcnt1, sdir(1)/ALLcnt1, . . . , sdir(29)/A LLcnt1}
hist1_P1[f][30]={sdir(0)/ALLcnt1, sdir(1)/ALLcnt1, . . . , sdir(29)/A LLcnt1}

Similarly, the feature amount extraction unit 3 obtains hist1_P2[g][30] through hist1_P2[l][30] corresponding to division pattern P2, and performs a normalization process on each of histograms hist1_P2[g][30]˜hist1_P2[l][30], and makes the storage unit 5 store normalized histograms hist1_P2[g][30] through hist1_P2[l][30] as the above matching feature amounts or the above registered feature amounts (first feature amount).

Next, the matching unit 7 treats, as score11, the sum of the absolute value of a difference between hist1_P1[a][30] as a registered feature amount and hist1_P1[a][30] as a matching feature amount, the absolute value of a difference between hist1_P1[b][30] as a registered feature amount and hist1_P1[b][30] as a matching feature amount, the absolute value of a difference between hist1_P1[c][30] as a registered feature amount and hist1_P1[c][30] as a matching feature amount, the absolute value of a difference between hist1_P1[d][30] as a registered feature amount and hist1_P1[d][30] as a matching feature amount, the absolute value of a difference between hist1_P1[e][30] as a registered feature amount and hist1_P1[e][30] as a matching feature amount, and the absolute value of a difference between hist1_P1[f][30] as a registered feature amount and hist1_P1[f][30] as a matching feature amount.

Also, the matching unit 7 treats, as score12, the sum of the absolute value of a difference between hist1_P2[g][30] as a registered feature amount and hist1_P2[g][30] as a matching feature amount, the absolute value of a difference between hist1_P2[h][30] as a registered feature amount and hist1_P2[h][30] as a matching feature amount, the absolute value of a difference between hist1_P2[i][30] as a registered feature amount and hist1_P2[i][30] as a matching feature amount, the absolute value of a difference between hist1_P2[j][30] as a registered feature amount and hist1_P2[j][30] as a matching feature amount, the absolute value of a difference between hist1_P2[k][30] as a registered feature amount and hist1_P2[k][30] as a matching feature amount, and the absolute value of a difference between hist1_P2[l][30] as a registered feature amount and hist1_P2[l][30] as a matching feature amount.

Then, the matching unit 7 treats the sum of score11 and score12 as score shown in FIG. 8.

After narrowing down a plurality of pieces of registered vein data, the biometrics authentication device 6 of an embodiment of the present disclosure has obtained the degrees of similarity between the narrowed-down pieces of registered vein data and the matching vein data, making it possible to suppress an increase in the matching process time compared with a case of obtaining the degrees of similarity between all of the plurality of pieces of registered vein data and the matching vein data.

Also, the biometrics information registration device 1 and the biometrics authentication device 6 according to an embodiment of the present disclosure obtain the angle between two of respective divisional segments among a plurality of segments obtained by vectorizing a vein image for extracting feature amounts from an image for each of all combinations between the two segments, and treats the histograms of these angles as the feature amount (first feature amount), making it possible to reduce variation in the feature amount even when the photography environment or the orientation of the user has changed between the registration of the vein data and the authentication, thereby making it possible to extract a registered feature amount and a matching feature amount highly accurately. This also makes it possible to suppress a decrease in the accuracy of the authentication process.

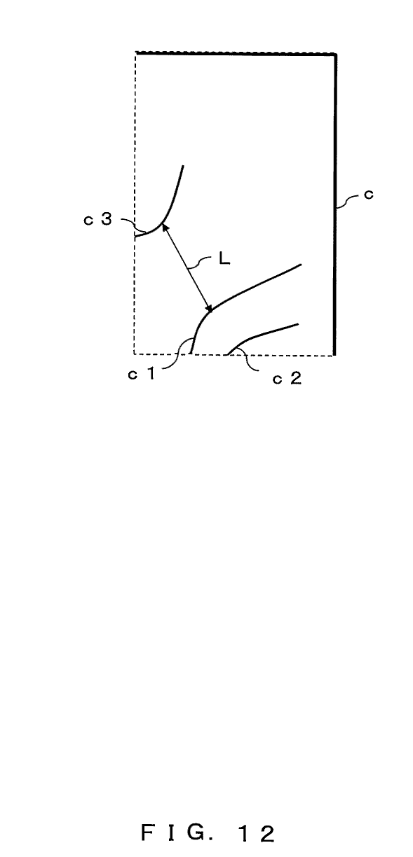

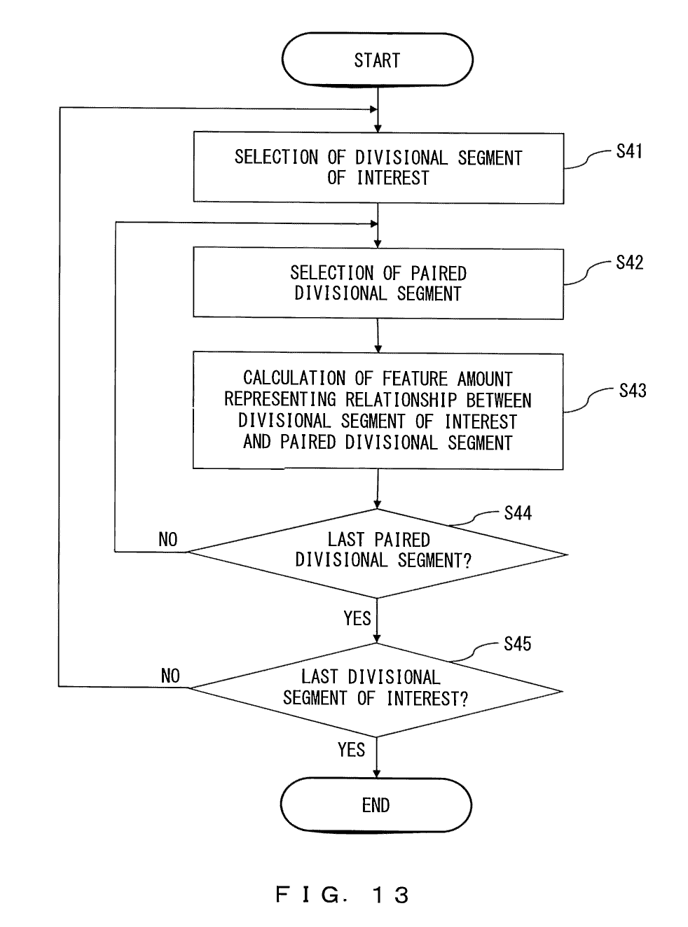

Incidentally, when the histogram of an angle between two segments is treated as a feature amount and the angle between the two segments shown in FIG. 15A and the angle between the two segments shown in FIG. 15B are equal, the two segments shown in FIG. 15A and the two segments shown in FIG. 15B are determined to be equal.

Then, it is possible to obtain the direction of an angle between two segments and treat the histogram of that direction (frequency distribution) as a feature amount.

Explanations will be given for a method of calculating a feature amount when the histogram of the direction of an angle between two segments is treated as that feature amount (second feature amount).

It is assumed for example that the image has been divided by division pattern P1, area c has been selected as shown in FIG. 11A, segment c1 has been selected as segment of interest c1 and segment c2 has been selected as paired segment c2 as shown in FIG. 12.

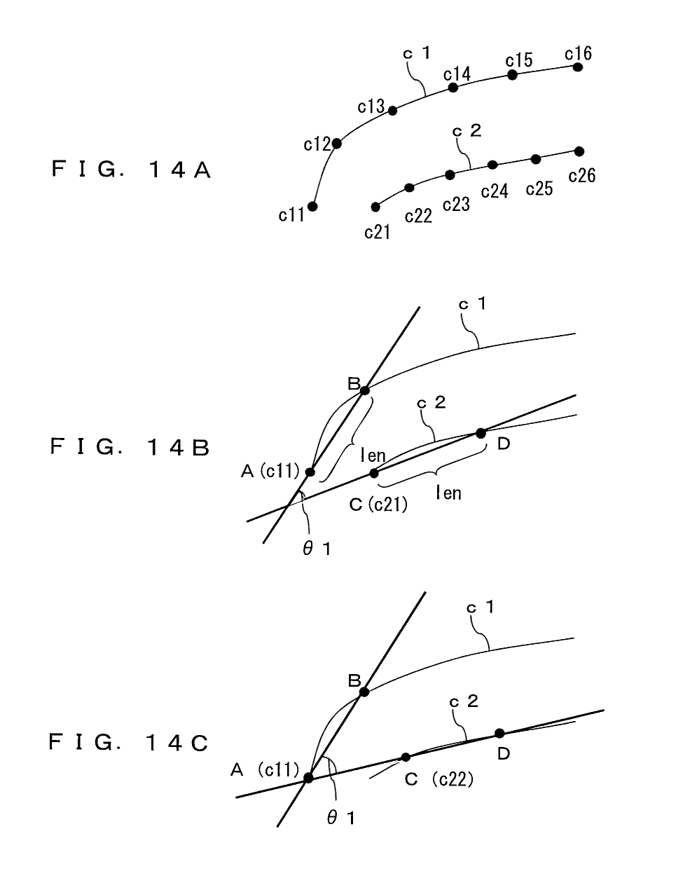

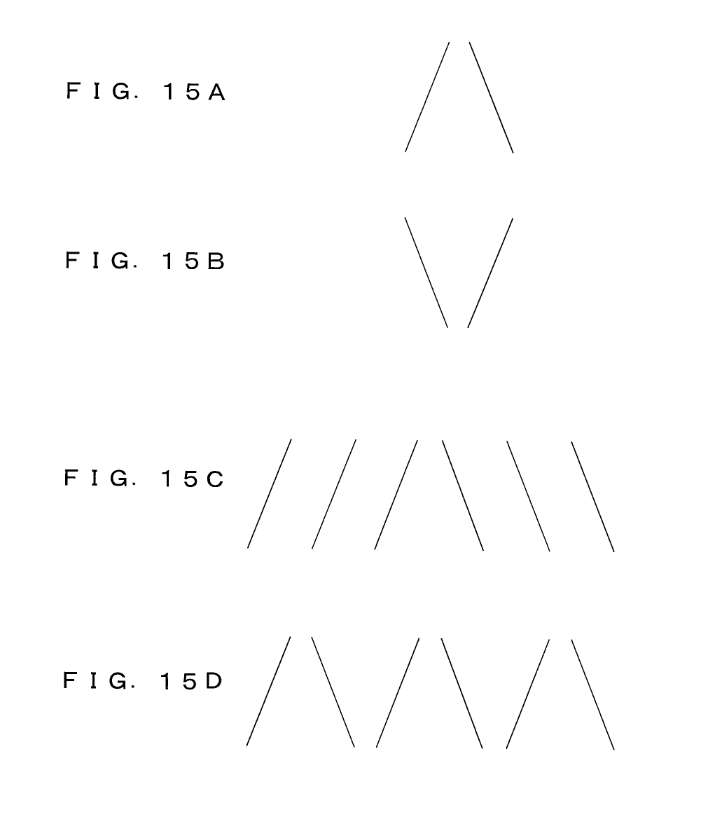

First, the feature amount extraction unit 3 obtains all end points and inflection points of segment of interest c1, treats these points as points c11 through c16 as shown in FIG. 16A, treats point c11 as point A, treats as point B a point distant from point A on segment of interest c1 by linear distance len, and treats as divisional segment of interest AB straight line AB passing through points A and B as shown in FIG. 16B.

Next, the feature amount extraction unit 3 obtains all end points and inflection points of paired segment c2, treats these points as points c21 through c26 as shown in FIG. 16A, treats point c21 as point C, treats as point D a point distant from point C on paired segment c2 by linear distance len, and treats as paired divisional segment CD straight line CD passing through points C and D as shown in FIG. 16B.

Next, the feature amount extraction unit 3 obtains direction θ2 of an angle between divisional segment of interest AB and paired divisional segment CD. Specifically, the feature amount extraction unit 3 translates divisional segment of interest AB and paired divisional segment CD in such a manner that points A and C coincide with the origin of the two-dimensional coordinate system as shown in FIG. 16C, draws a perpendicular line from the origin to straight line BD that passes through translated points B and D, treats the coordinate position of intersection H between that perpendicular line and straight line BD as (Hx,Hy), and calculates θ2=a tan 2(Hy,Hx)*(180/π) so as to obtain rotation angle θ2 from the Hx axis to the perpendicular line in the two-dimensional coordinate system having the origin at its center, and treats rotation angle θ2 as direction θ2 of the angle between divisional segment of interest AB and paired divisional segment CD.

Next, the feature amount extraction unit 3 obtains hist2_P1[Area][n] as a histogram (frequency distribution) for direction θ2. Note that P1 represents division pattern P1, [Area] represents an area after the division of the image, and [n] represents the number of grades of a histogram. For example, when the angle of 360 degrees is partitioned in units of 8 degrees so as to set 45 angle regions (grades), the counter value of an angle region including direction θ2 is incremented from among counter values ddir(0) through ddir(44) respectively corresponding to the 45 angle regions, and obtains hist2_P1[c][45]={ddir(0), ddir(1), . . . , ddir(44)} as a histogram. When for example direction θ2 calculated then is 15 degrees, the feature amount extraction unit 3 increments counter value ddir(1), which corresponds to the angle region from 8 degrees to 15 degrees, so as to obtain hist2_P1[c][45]={0, 1, 0, . . . , 0}.

Next, the feature amount extraction unit 3 treats next point c22 as point C, treats as point D a point distant from point C on paired segment c2 by linear distance len, and treats straight line CD passing through points C and D as next paired divisional segment CD.

Next, the feature amount extraction unit 3 calculates direction θ2 of the angle between divisional segment of interest AB and paired divisional segment CD.

Next, the feature amount extraction unit 3 increments the counter value of the angle region including that direction θ2 and obtains hist2_P1[c][45]={ddir(0), ddir(1), . . . , ddir(44)} as a histogram. When for example direction θ2 calculated then is 23 degrees, the feature amount extraction unit 3 increments counter value ddir(2) corresponding to the angle region from 16 degrees to 23 degrees so as to obtain hist2_P1[c][45]={0, 1, 1, 0, . . . , 0}.

As described above, direction θ2 of the angle between divisional segment of interest AB and paired divisional segment CD is repeatedly calculated by referring to point c11 until it becomes impossible to select paired divisional segment CD in paired segment c2 and hist2_P1[c][45]={ddir(0), ddir(1), . . . , ddir(44)} is obtained as a histogram for each of such directions 02.

Next, the feature amount extraction unit 3 treats next point c12 as point A, treats as point B a point distant from point A on segment of interest c1 by linear distance len, treats as next divisional segment of interest AB straight line AB passing through points A and B, treats point c21 as point C, treats as point D a point distant from point C on paired segment c2 by linear distance len, and treats straight line CD passing through points C and D as paired divisional segment CD.

Next, the feature amount extraction unit 3 calculates direction θ2, increments the counter value of the angle region including that direction θ2, and obtains hist2_P1[c][45]={ddir(0), ddir(1), . . . , ddir(44)}.

As described above, direction θ2 is obtained for each of all the combinations between selectable divisional segments of interest AB and selectable paired divisional segments CD so as to obtain hist2_P1[c][45]={ddir(0), ddir(1), . . . , ddir(44)} for that direction θ2.

Next, the feature amount extraction unit 3 similarly obtains hist2_P1[a][45], hist2_P1[b][45], hist2_P1[d][45], hist2_P1[e][45] and hist2_P1[f][45] as histograms for other areas a, b, d, e and f of the image shown in FIG. 11A.

Then, the feature amount extraction unit 3 performs a normalization process on each of hist2_P1[a][45] through hist2_P1[f][45]. For example, the feature amount extraction unit 3 treats, as ALLcnt1, the sum of the total of ddir(0), ddir(1), . . . , ddir(44) of histogram hist2_P1[a][45], the total of ddir(0), . . . , ddir(1), . . . , ddir(44) of hist2_P1[b][45], . . . , and the total of ddir(0), ddir(1), . . . , ddir(44) of hist2_P1[f][45], expresses the ratios of the counter values by dividing each counter value by sum ALLcnt1 in an integer as described below so as to perform a normalization process, and makes the storage unit 5 store normalized histograms hist2_P1[a][45] through hist2_P1[f][45] as the above matching feature amounts or the above registered feature amounts (second feature amount).

hist2_P1[a][45]={ddir(0)/ALLcnt1, ddir(1)/ALLcnt1, . . . , ddir(44)/A LLcnt1}
hist2_P1[b][45]={ddir(0)/ALLcnt1, ddir(1)/ALLcnt1, . . . , ddir(44)/A LLcnt1}
hist2_P1[c][45]={ddir(0)/ALLcnt1, ddir(1)/ALLcnt1, . . . , ddir(44)/A LLcnt1}
hist2_P1[d][45]={ddir(0)/ALLcnt1, ddir(1)/ALLcnt1, . . . , ddir(44)/A LLcnt1}
hist2_P1[e][45]={ddir(0)/ALLcnt1, ddir(1)/ALLcnt1, . . . , ddir(44)/A LLcnt1}
hist2_P1[f][45]={ddir(0)/ALLcnt1, ddir(1)/ALLcnt1, . . . , ddir(44)/A LLcnt1}

Similarly, the feature amount extraction unit 3 obtains hist2_P2[g][45] through hist2_P2[l][45] corresponding to division pattern P2, and performs a normalization process on each of histograms hist2_P2[g][45] through hist2_P2[l][45], and makes the storage unit 5 store normalized histograms hist2_P2[g][45] through hist2_P2[l][45] as the above matching feature amounts or the above registered feature amounts (second feature amount).

Next, the matching unit 7 treats, as score21, the sum of the absolute value of a difference between hist2_P1[a][45] as a registered feature amount and hist2_P1[a][45] as a matching feature amount, the absolute value of a difference between hist2_P1[b][45] as a registered feature amount and hist2_P1[b][45] as a matching feature amount, the absolute value of a difference between hist2_P1[c][45] as a registered feature amount and hist2_P1[c][45] as a matching feature amount, the absolute value of a difference between hist2_P1[d][45] as a registered feature amount and hist2_P1[d][45] as a matching feature amount, the absolute value of a difference between hist2_P1[e][45] as a registered feature amount and hist2_P1[e][45] as a matching feature amount, and the absolute value of a difference between hist2_P1[f][45] as a registered feature amount and hist2_P1[f][45] as a matching feature amount.

Next, the matching unit 7 treats, as score22, the sum of the absolute value of a difference between hist2_P2[g][45] as a registered feature amount and hist2_P2[g][45] as a matching feature amount, the absolute value of a difference between hist2_P2[h][45] as a registered feature amount and hist2_P2[h][45] as a matching feature amount, the absolute value of a difference between hist2_P2[i][45] as a registered feature amount and hist2_P2[i][45] as a matching feature amount, the absolute value of a difference between hist2_P2[j][45] as a registered feature amount and hist2_P2[j][45] as a matching feature amount, the absolute value of a difference between hist2_P2[k][45] as a registered feature amount and hist2_P2[k][45] as a matching feature amount, and the absolute value of a difference between hist2_P2[l][45] as a registered feature amount and hist2_P2[l][45] as a matching feature amount.

Then, the matching unit 7 calculates score=α×(score11+score12)+β×(score21+score22) so as to obtain score shown in FIG. 8. Note that α and β are weight coefficients.

Also, other feature amounts may be used for obtaining score.

FIG. 17 explains a third feature amount.

First, the feature amount extraction unit 3 removes a thin vein image and a thick vein image from an image obtained by the image obtainment unit 2, and thereafter develops such vein images onto the center of an image of a different size (an image of 256 by 256 for example), and treats that image as f(x,y).

Next, the feature amount extraction unit 3 performs two-dimensional fast Fourier transform on image f(x,y) as expressed by expression 1 so as to obtain spatial frequency component F(u,v). This two-dimensional fast Fourier transform first performs Fourier transform on pixels of each line in the direction of x of image f(x,y), and thereafter performs Fourier transform on the transform result of that line in the direction of y. It is assume that W1=exp(−j2π/M) and W2=exp(−j2π/N) are satisfied, where M and N represent the numbers of pixels in the horizontal and vertical directions, respectively.

$$F ( u , v ) = \frac{1}{MN} {\sum }_{m = 0}^{M - 1} {\sum }_{n = 0}^{N - 1} f ( x , y ) {W}_{1}^{mn} {W}_{2}^{nv} \qquad Expression 1$$

Next, the feature amount extraction unit 3 calculates P(u,v)=|F(u,v)|² so as to obtain power spectrum P(u,v).

Next, the feature amount extraction unit 3 treats power spectrum P(u,v) as power spectrum P(r,θ) of the polar coordinate format and conducts the operation as expressed by expression 2 so as to obtain energy p′(r) in the a doughnut-shaped region having the origin at its center. Note that θ is in the range from 0 through π.

$${p}^{′} ( r ) = {\sum }_{θ = 0}^{π} p ( r , θ ) \qquad Expression 2$$

Then, the feature amount extraction unit 3 calculates p(r)=10000*p′(r)/Σp′(r) so as to obtain, as the third feature amount, the energy ratio P(r) of each frequency. Note that “r” represents the radius, which satisfies r=1 through 32 in this example, and “10000” is a correction value for integer-type transform.

As described above, the third feature amount represents directionality and an amount of a vein image by using a frequency component, and can be expressed by the sum of energy in doughnut-shaped regions around the origin in the polar coordinate system power spectrum space as shown in FIG. 17. In this example, as the above matching feature amount or the above registered feature amount (third feature amount), the storage unit 5 stores {p(1), p(2), . . . , p(32)} for 32 frequency components in a case when radius r has been changed from 1 through 32.

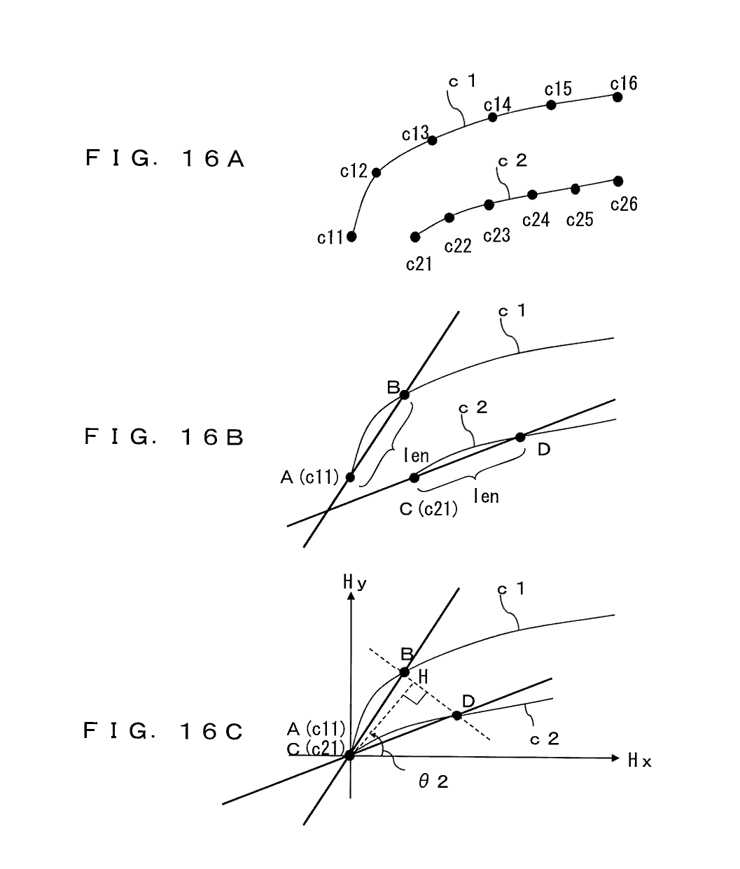

FIG. 18 explains a fourth feature amount.

First, similarly to the third feature amount, the feature amount extraction unit 3 performs Fourier transform on image f(x,y) so as to calculate spatial frequency component F(u,v), and calculates power spectrum P(u,v) from this spatial frequency component F(u,v).

Next, the feature amount extraction unit 3 treats power spectrum P(u,v) as power spectrum P(r,θ) of the polar coordinate format and conducts operations as expressed by expression 3 so as to obtain the energy of the angle. Note that w is the size of the domain of definition of P(u,v) and θ represents the directions obtained by dividing 180 degrees by 12.

$${q}^{′} ( 0 ) = {\sum }_{θ = 0}^{w / 2} q ( r , θ ) \qquad Expression 3$$

Then, the feature amount extraction unit 3 calculates q(θ)=10000*q′ (θ)/Σq′ (θ) so as to obtain, as the fourth feature amount, energy ratio q(θ) of each angle. In other words, the energy ratio in each angle range obtained by the division by 12 is calculated. Note that “10000” is a correction value for an integer-type transform.

As described above, the fourth feature amount represents directionality and an amount of a vein image by using an angle component, and can be expressed by the sum of energy in angle ranges each of which is of 15 degrees as shown in FIG. 18. In this example, as the above matching feature amount or the above registered feature amount (fourth feature amount), the storage unit 5 stores {q(0), q(1), . . . , q(11)} for 12 angle components in a case when angle θ has been changed from 0 through 180.

Note that angle component q(0) is the energy of the angle with θ ranging from 0 through 14, and thus is calculated by expression 4, and is calculated for the 12 directions by changing θ sequentially.

$$q ( 0 ) = {\sum }_{θ = 0}^{14} q ( θ ) \qquad Expression 4$$

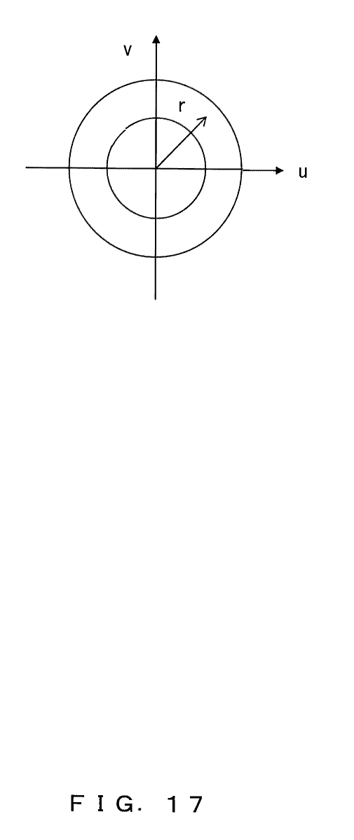

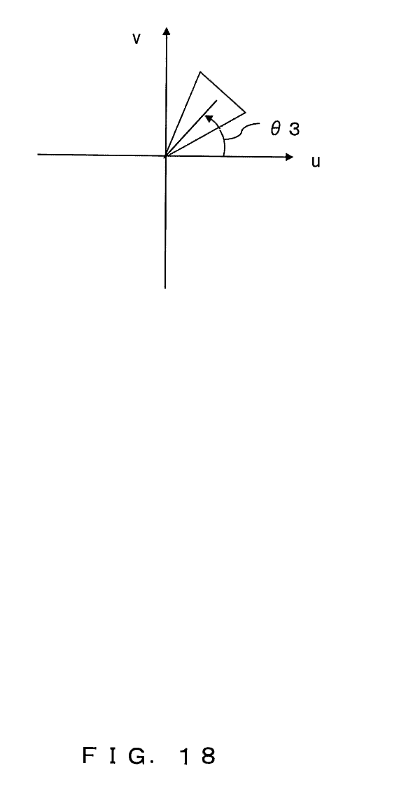

FIG. 19 explains a fifth feature amount.

First, the feature amount extraction unit 3 divides the image by division pattern P1 for example as shown in FIG. 11A, and selects one area.

Next, the feature amount extraction unit 3 obtains all curvature directions of two divisional segments adjacent in a segment obtained by vectorizing a vein image in the selected area, and obtains the fifth feature amount, which represents the histogram (frequency distribution) of those curvature directions. For example, the feature amount extraction unit 3 obtains all end points and inflection points on segment c1 as shown in FIG. 19A so as to treat these points as points c11 through c16, treats point c11 as point A, treats as point B a point distant from point A on segment c1 by linear distance len as shown in FIG. 19B, and treats straight line AB passing through points A and B as divisional segment AB. Next, the feature amount extraction unit 3 treats as point c a point distant from point B on segment of interest c1 by linear distance len as shown in FIG. 19B, and treats straight line CD passing through points B and C as divisional segment CD. Next, the feature amount extraction unit 3 treats coordinates A, B and C obtained by translating coordinates A, B and C in such a manner that coordinates B become the origin as A(xa,ya), B(xb,yb) and C(xc,yc), treats the coordinates of the intersection between straight line AC and the perpendicular line from point B therefor as H(Hx,Hy), calculates coordinates H(Hx,Hy) by expressions 5 and 6, and calculates θ4=a tan 2(Hy,Hx)*(180/π), and thereby obtains curvature direction θ4 of the angle between divisional segment AB and divisional segment CD.

$$\begin{matrix}Hx = - \frac{( xc * ya - xa * yc )}{{( xc - xa )}^{2} + {( yc - ya )}^{2}} * ( yc - ya ) & Expression 5 \\ Hy = \frac{( xc * ya - xa * yc )}{{( xc - xa )}^{2} + {( yc - ya )}^{2}} * ( xc - xa ) & Expression 6\end{matrix}$$

Next, the feature amount extraction unit 3 obtains hist3_P1[Area][n] as a histogram for curvature direction θ4. Note that P1 represents division pattern P1, [Area] represents an area after the division of the image, and [n] represents the number of grades of a histogram. For example, when area c is selected and the angle of 360 degrees is partitioned in units of 10 degrees so as to set 36 angle regions (grades), the counter value of an angle region including curvature direction θ4 is incremented from among counter values curv(0) through curv(35) respectively corresponding to the 36 angle regions, and obtains hist3_P1[c][36]={curv(0), curv(1), . . . , curv(35)} as a histogram. When for example direction θ1 calculated then is 330 degrees, the feature amount extraction unit 3 increments counter value curv(33), which corresponds to the angle region from 330 degrees to 339 degrees, so as to obtain hist3_P1[c][36]={0, . . . , 0, 1, 0, 0}. Note that curv(0) represents a value obtained by integrating curvature direction θ4 included in the angle region from 0 degree to 9 degrees, and is calculated by expression 7.

$$curv ( 0 ) = {\sum }_{θ4 = 0}^{10 - 1} curv ( θ4 ) \qquad Expression 7$$

Next, as shown in FIG. 19D, the feature amount extraction unit 3 treats next point c12 as point A, treats as point B a point distant from point A on segment c1 by linear distance len, and treats straight line AB passing through points A and B as divisional segment AB.

Next, as shown in FIG. 19D, the feature amount extraction unit 3 treats as point C a point distant from point B on segment of interest c1 by linear distance len, and selects straight line BC passing through points B and C as divisional segment BC.

As described above, curvature direction θ4 is repeatedly calculated until it becomes impossible to select divisional segment AB in segment c1 and hist3_P1[c][36]={curv(0), curv(1), . . . , curv(35)} is obtained as a histogram for each of such curvature directions 04.

The generation of this histogram hist3_P1[c][36] is conducted similarly for other areas a, b, d, e and f.

Then, the feature amount extraction unit 3 treats, as ALLcnt1, the sum of the total of curv(0), curv(1), . . . , curv(35) of histogram hist3_P1[a][36], the total of curv(0), curv(1), . . . , curv(35) of hist3_P1[b][36], . . . , and the total of curv(0), curv(1), . . . , curv(35) of hist3_P1[f][36], expresses the ratios of the counter values by dividing each counter value by sum ALLcnt1 in an integer as described below so as to perform a normalization process, and makes the storage unit 5 store normalized histograms hist3_P1[a][36] through hist3_P1[f][36] as the above matching feature amounts or the above registered feature amounts (fifth feature amount).

hist3_P1[a][36]={curv(0)/ALLcnt1, curv(1)/ALLcnt1, . . . , curv(35)/A LLcnt1}
hist3_P1[b][36]={curv(0)/ALLcnt1, curv(1)/ALLcnt1, . . . , curv(35)/A LLcnt1}
hist3_P1[c][36]={curv(0)/ALLcnt1, curv(1)/ALLcnt1, . . . , curv(35)/A LLcnt1}
hist3_P1[d][36]={curv(0)/ALLcnt1, curv(1)/ALLcnt1, . . . , curv(35)/A LLcnt1}
hist3_P1[e][36]={curv(0)/ALLcnt1, curv(1)/ALLcnt1, . . . , curv(35)/A LLcnt1}
hist3_P1[f][36]={curv(0)/ALLcnt1, curv(1)/ALLcnt1, . . . , curv(35)/A LLcnt1}

Similarly, the feature amount extraction unit 3 obtains hist3_P2[g][36] through hist3_P2[l][36] corresponding to division pattern P2, and performs a normalization process on each of histograms hist3_P2[g][36] through hist3_P2[l][36], and makes the storage unit 5 store normalized histograms hist3_P2[g][36] through hist3_P2[l][36] as the above matching feature amounts or the above registered feature amounts (fifth feature amount).

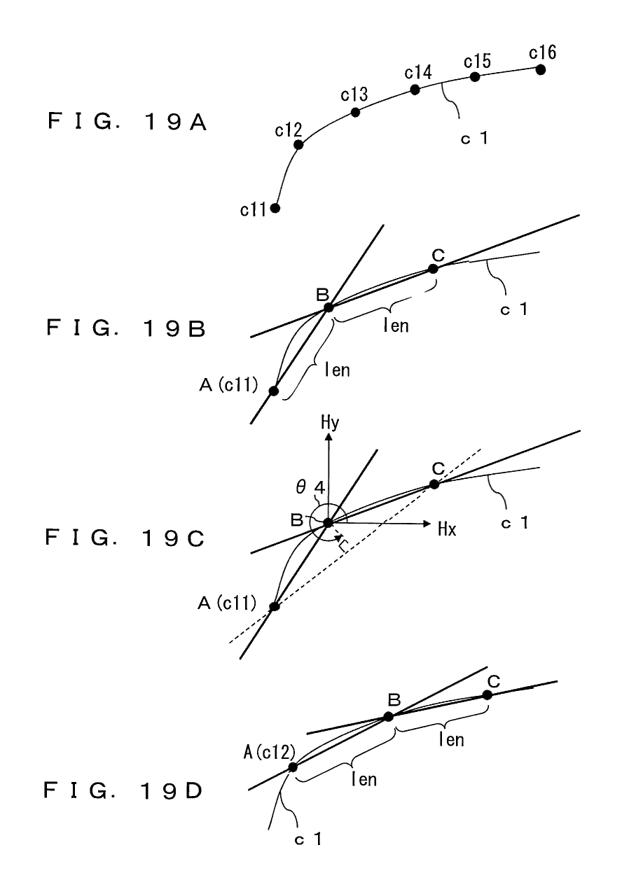

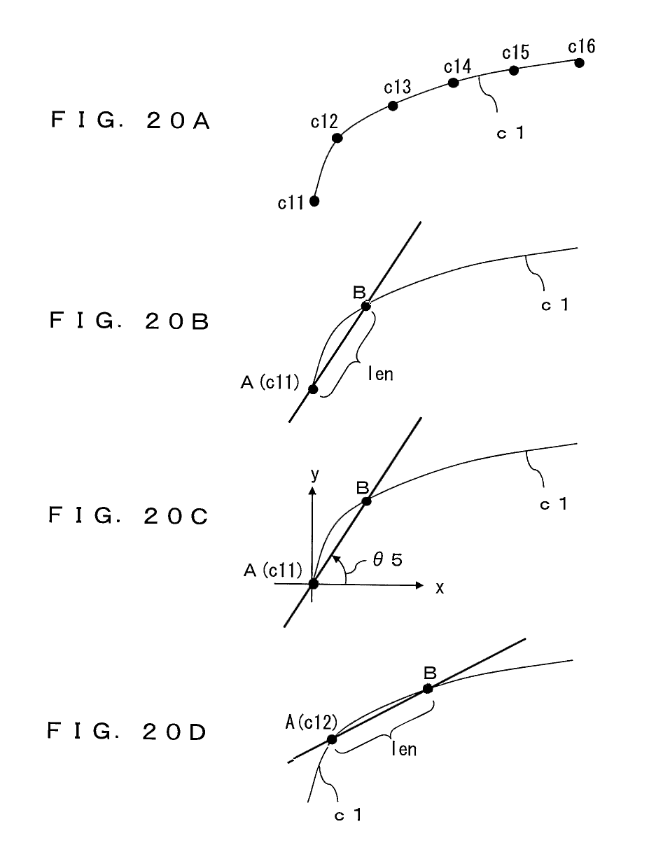

FIG. 20 explains a sixth feature amount.

First, the feature amount extraction unit 3 divides the image by division pattern P1 for example as shown in FIG. 11A, and selects one area.

Next, the feature amount extraction unit 3 obtains inclinations of all divisional segments in a segment obtained by vectorizing a vein image in the selected area, and obtains the sixth feature amount, which represents the histogram (frequency distribution) of those inclinations. For example, the feature amount extraction unit 3 obtains all end points and inflection points on segment c1 as shown in FIG. 20A so as to treat these points as points c11 through c16, treats point c11 as point A, treats as point B a point distant from point A on segment c1 by linear distance len, and treats straight line AB passing through points A and B as divisional segment AB as shown in FIG. 20B. Next, the feature amount extraction unit 3 treats the coordinates of points A and B as A(xa,ya) and B(xb,yb) so as to calculate θ5=a tan 2(yb−ya,xb−xa)*(180/π), and thereby obtain inclination θ5 of divisional segment AB. When the angle is a negative value, 180 degrees are added and when the angle is equal to or greater than 180 degrees, 180 degrees are subtracted. The purpose of this is to consider that the inclination of 270 degrees or −90 degrees is identical to that of 90 degrees. Next, the feature amount extraction unit 3 obtains histogram hist4_P1[Area][n] for inclination θ5. Note that P1 represents division pattern P1, [Area] represents an area after the division of the image, and [n] represents the number of grades of a histogram. For example, when area c is selected and the angle of 180 degrees is partitioned in units of 10 degrees so as to set 18 angle regions (grades), the counter value of an angle region including direction θ5 is incremented from among counter values segdir(0) through segdir(17) respectively corresponding to the 18 angle regions, and obtains hist4_P1[c][18]={segdir(0), segdir(1), . . . , segdir(17)} as a histogram. When for example inclination θ5 calculated then is 45 degrees, the feature amount extraction unit 3 increments counter value segdir(4), which corresponds to the angle region from 40 degrees to 49 degrees, so as to obtain hist4_P1[c][18]={0, 0, 0, 0, 1, 0, . . . , 0}. Note that segdir(0) represents a value obtained by integrating inclination θ5 included in the angle region from 0 degrees to 9 degrees, and is calculated by expression 8.

$$segdir ( 0 ) = {\sum }_{θ5 = 0}^{10 - 1} segdir ( θ5 ) \qquad Expression 8$$

Next, as shown in FIG. 20D, the feature amount extraction unit 3 treats next point c12 as point A, treats as point B a point distant from point A on segment c1 by linear distance len, and treats straight line AB passing through points A and B as divisional segment AB.

As described above, inclination θ5 is repeatedly calculated until it becomes impossible to select divisional segment AB in segment c1 and hist4_P1[c][18]={segdir(0), segdir(1), . . . , segdir(17)} is obtained as a histogram for each of such inclinations 05.

The generation of this histogram hist4_P1[c][18] is conducted similarly for other areas a, b, d, e and f.

Then, the feature amount extraction unit 3 treats, as ALLcnt1, the sum of the total of segdir(0), segdir(1), . . . , segdir(17) of histogram hist4_P1[a][18], the total of segdir(0), segdir(1), . . . , segdir(17) of hist4_P1[b][18], . . . , and the total of segdir(0), segdir(1), . . . , segdir(17) of hist4_P1[f][18], expresses the ratios of the counter values by dividing each counter value by sum ALLcnt1 in an integer as described below so as to perform a normalization process, and makes the storage unit 5 store normalized histograms hist4_P1[a][18] through hist4_P1[f][18] as the above matching feature amounts or the above registered feature amounts (sixth feature amount).

hist4_P1[a][18]={segdir(0)/ALLcnt1, segdir(1)/ALLcnt1, . . . , segdir (17)/ALLcnt1}
hist4_P1[b][18]={segdir(0)/ALLcnt1, segdir(1)/ALLcnt1, . . . , segdir (17)/ALLcnt1}
hist4_P1[c][18]={segdir(0)/ALLcnt1, segdir(1)/ALLcnt1, . . . , segdir (17)/ALLcnt1}
hist4_P1[d][18]={segdir(0)/ALLcnt1, segdir(1)/ALLcnt1, . . . , segdir (17)/ALLcnt1}
hist4_P1[e][18]={segdir(0)/ALLcnt1, segdir(1)/ALLcnt1, . . . , segdir (17)/ALLcnt1}
hist4_P1[f][18]={segdir(0)/ALLcnt1, segdir(1)/ALLcnt1, . . . , segdir (17)/ALLcnt1}

Similarly, the feature amount extraction unit 3 obtains hist4_P2[g][18] through hist4_P2[l][18] corresponding to division pattern P2, and performs a normalization process on each of histograms hist4_P2[g][18] through hist4_P2[l][18], and makes the storage unit 5 store normalized histograms hist4_P2[g][18] through hist4_P2[l][18] as the above matching feature amounts or the above registered feature amounts (sixth feature amount).

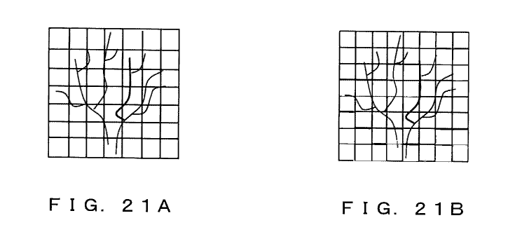

FIG. 21A and FIG. 21B explain a seventh feature amount.

As shown in for example FIG. 21A, the feature amount extraction unit 3 first divides an image obtained by the image obtainment unit 2 into 49 (=7 by 7) areas by division pattern P1 as shown in FIG. 21A, and also divides the image obtained by the image obtainment unit 2 into 64 (=8 by 8) areas by division pattern P2 as shown in FIG. 21B. Note that the division patterns are not limited to those shown in FIG. 21A or FIG. 21B.

Next, the feature amount extraction unit 3 obtains the number of pixels seghist1 corresponding to a vein image in each of the 49 areas obtained by the division based on division pattern P1.

Next, the feature amount extraction unit 3 generates hist5_P1[49]={seghist1(0), seghist1(1), . . . , seghist1(48)} as a histogram for the number of pixels seghist1 in a vein image in each area.

Next, the feature amount extraction unit 3 treats, as ALLcnt1, the sum of seghist1(0), seghist1(1), . . . , seghist1(48) of histogram hist5_P1[49], expresses the ratios of the counter values by dividing each counter value by sum ALLcnt1 in an integer as described below so as to perform a normalization process, and makes the storage unit 5 store normalized histogram hist5_P1[49] as the above matching feature amounts or the above registered feature amounts (seventh feature amount).

hist5_P1[49]={seghist1(0)/ALLcnt1, seghist1(1)/ALLcnt1, . . . , seghist1(48)/ALLcnt1}

Similarly, the feature amount extraction unit 3 obtains hist5_P2[64]={seghist2(0), seghist2(1), . . . , seghist2(63)} as a histogram corresponding to division pattern P2, and performs a normalization process on that histogram hist5_P2[64], and makes the storage unit 5 store normalized histogram hist5_P2[64] as the above matching feature amounts or the above registered feature amounts (seventh feature amount).

Next, the matching unit 7 treats, as score3, the sum of the absolute value of a difference between p(1) as a registered feature amount and p(1) as a matching feature amount, the absolute value of a difference between p(2) as a registered feature amount and p(2) as a matching feature amount, . . . , and the absolute value of a difference between p(32) as a registered feature amount and p(32) as a matching feature amount.

Also, the matching unit 7 treats, as score4, the sum of the absolute value of a difference between q(0) as a registered feature amount and q(0) as a matching feature amount, the absolute value of a difference between q(1) as a registered feature amount and q(1) as a matching feature amount, . . . , and the absolute value of a difference between q(11) as a registered feature amount and q(11) as a matching feature amount.

Next, the matching unit 7 treats, as score51, the sum of the absolute value of a difference between hist3_P1[a][36] as a registered feature amount and hist3_P1[a][36] as a matching feature amount, the absolute value of a difference between hist3_P1[b][36] as a registered feature amount and hist3_P1[b][36] as a matching feature amount, the absolute value of a difference between hist3_P1[c][36] as a registered feature amount and hist3_P1[c][36] as a matching feature amount, the absolute value of a difference between hist3_P1[d][36] as a registered feature amount and hist3_P1[d][36] as a matching feature amount, the absolute value of a difference between hist3_P1[e][36] as a registered feature amount and hist3_P1[e][36] as a matching feature amount, and the absolute value of a difference between hist3_P1[ f][36] as a registered feature amount and hist3_P1[ f][36] as a matching feature amount.

Also, the matching unit 7 treats, as score52, the sum of the absolute value of a difference between hist3_P2[g][36] as a registered feature amount and hist3_P2[g][36] as a matching feature amount, the absolute value of a difference between hist3_P2[h][36] as a registered feature amount and hist3_P2[h][36] as a matching feature amount, the absolute value of a difference between hist3_P2[i][36] as a registered feature amount and hist3_P2[i][36] as a matching feature amount, the absolute value of a difference between hist3_P2[j][36] as a registered feature amount and hist3_P2[j][36] as a matching feature amount, the absolute value of a difference between hist3_P2[k][36] as a registered feature amount and hist3_P2[k][36] as a matching feature amount, and the absolute value of a difference between hist3_P2[l][36] as a registered feature amount and hist3_P2[l][36] as a matching feature amount.

Also, the matching unit 7 treats, as score61, the sum of the absolute value of a difference between hist4_P1[a][18] as a registered feature amount and hist4_P1[a][18] as a matching feature amount, the absolute value of a difference between hist4_P1[b][18] as a registered feature amount and hist4_P1[b][18] as a matching feature amount, the absolute value of a difference between hist4_P1[c][18] as a registered feature amount and hist4_P1[c][18] as a matching feature amount, the absolute value of a difference between hist4_P1[d][18] as a registered feature amount and hist4_P1[d][18] as a matching feature amount, the absolute value of a difference between hist4_P1[e][18] as a registered feature amount and hist4_P1[e][18] as a matching feature amount, and the absolute value of a difference between hist4_P1[f][18] as a registered feature amount and hist4_P1[f][18] as a matching feature amount.

Also, the matching unit 7 treats, as score62, the sum of the absolute value of a difference between hist4_P2[g][18] as a registered feature amount and hist4_P2[g][18] as a matching feature amount, the absolute value of a difference between hist4_P2[h][18] as a registered feature amount and hist4_P2[h][18] as a matching feature amount, the absolute value of a difference between hist4_P2[i][18] as a registered feature amount and hist4_P2[i][18] as a matching feature amount, the absolute value of a difference between hist4_P2[j][18] as a registered feature amount and hist4_P2[j][18] as a matching feature amount, the absolute value of a difference between hist4_P2[k][18] as a registered feature amount and hist4_P2[k][18] as a matching feature amount, and the absolute value of a difference between hist4_P2[l][18] as a registered feature amount and hist4_P2[l][18] as a matching feature amount.

Also, the matching unit 7 treats, as score71, the sum of the absolute value of a difference between seghist1(0) as a registered feature amount and seghist1(0) as a matching feature amount, the absolute value of a difference between seghist1(1) as a registered feature amount and seghist1(1) as a matching feature amount, . . . , and the absolute value of a difference between seghist1(48) as a registered feature amount and seghist1(48) as a matching feature amount.

Also, the matching unit 7 treats, as score72, the sum of the absolute value of a difference between seghist2(0) as a registered feature amount and seghist2(0) as a matching feature amount, the absolute value of a difference between seghist2(1) as a registered feature amount and seghist2(1) as a matching feature amount, . . . , and the absolute value of a difference between seghist2(63) as a registered feature amount and seghist2(63) as a matching feature amount.

Also, the matching unit 7 calculates score=α×(score11+score12)+β×(score21+score22)+γ(score3)+δ×(score3)+ε×(score4)+ζ×(score51+score52)+λ×(score61+score62)+μ×(score71+score72) so as to obtain score shown in FIG. 8. Note that α, β, γ, δ, ε, ζ, λ and μ are weight coefficients. It is not necessary to use all of the third through seventh feature amounts, and it is also possible to use only at least one feature amount from among the third through seventh feature amounts.

As described above, the biometrics information registration device 1 and the biometrics authentication device 6 of an embodiment of the present disclosure extract seven types of feature amounts (first through seventh feature amounts) from an image obtained by the image obtainment unit 2 so as to use these feature amounts for narrowing down pieces of registered vein data, and thereby can increase the accuracy of the narrowing down and can conduct the matching process more accurately.

Note that there is a possibility for example that the fourth and fifth feature amounts obtained for the segment shown in FIG. 15C will be respectively similar to the fourth and fifth feature amounts obtained for the segment shown in FIG. 15D, whereas it is possible to use the first and second feature amounts for distinguishing the feature amounts obtained for the segment shown in FIG. 15C from the feature amounts obtained for the segment obtained shown in FIG. 15D.

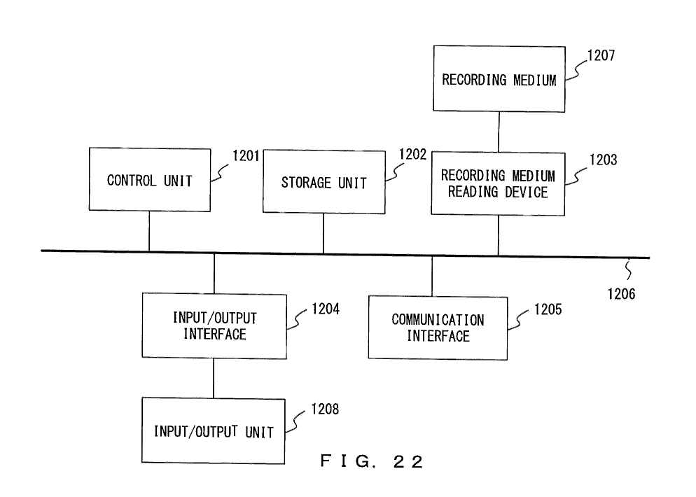

FIG. 22 shows an example of hardware constituting the biometrics information registration device 1 or the biometrics authentication device 6 of an embodiment of the present disclosure.

As shown in FIG. 22, the hardware constituting the biometrics information registration device 1 or the biometrics authentication device 6 includes a control unit 1201, a storage unit 1202, a recording medium reading device 1203, an input/output interface 1204, and a communication interface 1205, all of which are connected to each other via a bus 1206. Also, the hardware constituting the biometrics information registration device 1 or the biometrics authentication device 6 may be implemented by cloud computing etc.

The control unit 1201 may be implemented by for example a central processing unit (CPU), a multi-core CPU, a programmable device (field programmable gate array (FPGA), programmable logic device (PLD), etc.), and corresponds to the feature amount extraction unit 3 and the feature amount registration unit 4 shown in FIG. 1, and the matching unit 7 and the determination unit 8 shown in FIG. 6.

The storage unit 1202 corresponds to the storage unit 5 shown in FIG. 1 and FIG. 6, and may be implemented by for example a memory such as a read only memory (ROM), a random access memory (RAM), a hard disk, etc. Note that the storage unit 1202 may be used as a working area for execution. Also, another storage unit may be provided outside the biometrics information registration device 1 and the biometrics authentication device 6.

The recording medium reading device 1203 reads data stored in a recording medium 1207 and writes data to the recording medium 1207 under control of the control unit 1201. Also, recording medium 1207, which is removable, is a non-transitory computer-readable recording medium, and may be implemented by a magnetic recording device, an optical disk, a magneto-optical recording medium, a semiconductor memory, etc. A magnetic recording device may be implemented by for example a hard disk device (HDD) etc. An optical disk may be implemented by for example a digital versatile disk (DVD), a DVD-RAM, a compact disk read only memory (CD-ROM), a CD-R (Recordable)/RW (ReWritable), etc. A magneto-optical recording medium may be implemented by for example a magneto-optical (MO) disk etc. Also, a non-transitory recording medium includes the storage unit 1202.

To the input/output interface 1204, an input/output unit 1208 is connected, and the input/output interface 1204 transmits, to the control unit 1201 and via the bus 1206, information input by the user via the input/output unit 1208. Also, the input/output interface 1204 transmits, to the input/output unit 1208 and via the bus 1206, information transmitted from the control unit 1201.

The input/output unit 1208 corresponds to the image obtainment unit 2 shown in FIG. 1 and FIG. 6, and may be implemented by for example an image pickup device etc. Also, the input/output unit 1208 may be implemented by for example a keyboard, a pointing device (mouse etc.), a touch panel, a cathode ray tube (CRT) display, a printer, etc.

The communication interface 1205 is an interface for providing connection to a local area network (LAN) or to the Internet. Also, the communication interface 1205 can be used as an interface for providing LAN connection, Internet connection, or wireless connection to other computers, as needed.

By using a computer having the above hardware, the respective process functions performed by the biometrics information registration device 1 or the biometrics authentication device 6 are implemented. In such a case, by making a computer execute a program describing the contents of the respective process functions performed by the biometrics information registration device 1 or the biometrics authentication device 6, the above respective functions (for example, the feature amount extraction unit 3, the feature amount registration unit 4, the matching unit 7, and the determination unit 8) are implemented on the computer. The program describing the contents of the respective process functions can be stored in the storage unit 1202 or the recording medium 1207.

When the program is to be distributed, the program is sold in a state for example that it is stored in the recording medium 1207 such as a DVD, a CD-ROM, etc. It is also possible to record the program in a storage device of a server computer so that the program is transferred to another computer from the server computer via a network.

The computer that executes the program for example stores, in the storage unit 1202, the program recorded in the recording medium 1207 or the program transferred from the server computer. Then, the computer reads the program from the storage unit 1202 so as to execute a process in accordance with the program. Note that the computer may also read the program directly from the recording medium 1207 so as to execute a process in accordance with the program. Also, the computer may execute a process in accordance with a received program each time the program is transferred from the server program.

In the embodiments of the present disclosure, devices that conduct authentication by using a palm vein have been used for the explanations, whereas the scope of the present disclosure is not limited to this and any other portions from which biological features can be detected can be used.

For example, other portions from which biological features can be detected are not limited to veins, and may be a biological blood vessel image, a biological pattern, a biological fingerprint or palm pattern, the bottom of a foot, a toe, a finger, the back of a hand, the top of a foot, a wrist, an arm, etc.

Note that when a vein is used for the authentication, other portions from which biological features can be detected may be any portion that allows the observation of the vein.

Note that portions from which biological features can be detected and which can identify biological information are advantageous for authentication. For example, the palm of a hand or a face allows the identifying of the portion from the obtained image.

The embodiments of the present disclosure can suppress reduction in the authentication accuracy while suppressing an increase in the matching process time in 1:N authentication.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A biometrics information registration method for causing a computer to execute a process comprising:
extracting vein data representing a vein image and a feature amount from an image obtained by an image obtainment unit; and
making a storage unit store the vein data and the feature amount, wherein
the feature amount includes a first feature amount representing a relationship between two of a plurality of segments obtained by vectorizing the vein image.

2. The biometrics information registration method according to claim 1, wherein
the first feature amount represents a frequency distribution of angles obtained by obtaining an angle between divisional segments respectively of the two segments for each of all combinations of divisional segments respectively of the two segments.

3. The biometrics information registration method according to claim 2, wherein
the first feature amount is obtained for each of a plurality of areas when the image is divided into the plurality of areas by a first division pattern.

4. The biometrics information registration method according to claim 1, wherein
the feature amount includes a second feature amount representing a frequency distribution of directions obtained by obtaining a direction of an angle between divisional segments respectively of the two segments for each of all combinations of divisional segments respectively of the two segments.

5. The biometrics information registration method according to claim 4, wherein
the second feature amount is obtained for each of a plurality of areas when the image is divided into the plurality of areas by a second division pattern.

6. The biometrics information registration method according to claim 1, wherein
the feature amount includes:
a third feature amount representing directionality and an amount of the vein image by using a frequency component;
a fourth feature amount representing directionality and an amount of the vein image by using an angle component;
a fifth feature amount representing a frequency distribution of curvature directions obtained by obtaining all curvature directions of two divisional segments adjacent in the vein segment;
a sixth feature amount representing a frequency distribution of inclinations obtained by obtaining inclinations of all divisional segments in the segment; and
a seventh feature amount representing a frequency distribution of the numbers of pixels corresponding to the vein image obtained for each of a plurality of areas when the image is divided into the plurality of areas by a third division pattern.

7. A biometrics authentication method for causing a computer to execute a process comprising:
extracting vein data representing a vein image and a feature amount from an image obtained by an image obtainment unit;
narrowing down a plurality of pieces of registered vein data stored in the storage unit on the basis of a comparison result between the extracted feature amount and a registered feature amount stored in the storage unit, and obtaining a degree of similarity between the pieces of registered vein data that were narrowed down and the extracted vein data; and
determining whether or not a subject is a person to be authenticated, on the basis of the obtained degree of similarity, wherein
the feature amount includes a first feature amount representing a relationship between two of a plurality of segments obtained by vectorizing the vein image.

8. The biometrics authentication method according to claim 7, wherein
the first feature amount represents a frequency distribution of angles obtained by obtaining an angle between divisional segments respectively of the two segments for each of all combinations of divisional segments respectively of the two segments.

9. The biometrics authentication method according to claim 8, wherein
the first feature amount is obtained for each of a plurality of areas when the image is divided into the plurality of areas by a first division pattern.

10. The biometrics authentication method according to claim 7, wherein
the feature amount includes a second feature amount representing a frequency distribution of directions obtained by obtaining a direction of an angle between divisional segments respectively of the two segments for each of all combinations of divisional segments respectively of the two segments.

11. The biometrics authentication method according to claim 10, wherein
the second feature amount is obtained for each of a plurality of areas when the image is divided into the plurality of areas by a second division pattern.

12. The biometrics authentication method according to claim 7, wherein
the feature amount includes:
a third feature amount representing directionality and an amount of the vein image by using a frequency component;
a fourth feature amount representing directionality and an amount of the vein image by using an angle component;
a fifth feature amount representing a frequency distribution of curvature directions obtained by obtaining all curvature directions of two divisional segments adjacent in the vein segment;
a sixth feature amount representing a frequency distribution of inclinations obtained by obtaining inclinations of all divisional segments in the segment; and
a seventh feature amount representing a frequency distribution of the numbers of pixels corresponding to the vein image obtained for each of a plurality of areas when the image is divided into the plurality of areas by a third division pattern.

13. A biometrics information registration device comprising:
a feature amount extraction unit to extract vein data representing a vein image and a feature amount from an image obtained by an image obtainment unit; and
a feature amount registration unit to make a storage unit store the vein data and the feature amount extracted by the feature amount extraction unit, wherein
the feature amount includes a first feature amount representing a relationship between two of a plurality of segments obtained by vectorizing the vein image.

14. A biometrics authentication device comprising:
a feature amount extraction unit to extract vein data representing a vein image and a feature amount from an image obtained by an image obtainment unit;
a matching unit to narrow down a plurality of pieces of registered vein data stored in the storage unit on the basis of a comparison result between the feature amount extracted by the feature amount extraction unit and a registered feature amount stored in the storage unit, and obtaining a degree of similarity between the pieces of registered vein data that were narrowed down and the vein data extracted by the feature amount extraction unit; and
a determination unit to determine whether or not a subject is a person to be authenticated, on the basis of the degree of similarity obtained by the matching unit, wherein
the feature amount includes a first feature amount representing a relationship between two of a plurality of segments obtained by vectorizing the vein image.

15. A non-transitory computer-readable recording medium which records a program for causing a computer to execute a process comprising:
extracting vein data representing a vein image and a feature amount from an image obtained by an image obtainment unit; and
making a storage unit store the extracted vein data and feature amount, wherein
the feature amount includes a first feature amount representing a relationship between two of a plurality of segments obtained by vectorizing the vein image.

16. A non-transitory computer-readable recording medium which records a program for causing a computer to execute a process comprising:
extracting vein data representing a vein image and a feature amount from an image obtained by an image obtainment unit;
narrowing down a plurality of pieces of registered vein data stored in the storage unit on the basis of a comparison result between the extracted feature amount and a registered feature amount stored in the storage unit, and obtaining a degree of similarity between the pieces of registered vein data that were narrowed down and the vein data extracted by the feature amount extraction unit; and
determining whether or not a subject is a person to be authenticated, on the basis of the obtained degree of similarity, wherein
the feature amount includes a first feature amount representing a relationship between two of a plurality of segments obtained by vectorizing the vein image.
